(19) United States
(12) Patent Application Publication
(10) Pub. No.: US 2012/0096632 A1
(43) Pub. Date: Apr. 26, 2012

(54) SWIMMING GOGGLE AND FASTENING ASSEMBLY THEREOF

(75) Inventor: Shu-Fang CHOU, Zhonghe City (TW)

(73) Assignee: FIRST RANK CO., LTD.

(21) Appl. No.: 12/909,103

(22) Filed: Oct. 21, 2010

(51) Int. Cl. A61F 9/02

(52) U.S. Cl. 2/452

(57) ABSTRACT

A swimming goggle includes an eyepiece frame, a head strap and two fastening assemblies for connecting the eyepiece frame and the head strap. Both sides of the eyepiece frame have a through-hole respectively. Both ends of the head strap have protrusions. The fastening assembly includes a fastening base and a shroud assembled with the fastening base. The fastening base has an operating arm formed with an engaging piece. The fastening base has three tongues, a locking hole corresponding to the through-hole, and a trough for allowing the head strap to pass through. The shroud has a hook disposed through the through-hole and engaged with the locking hole. The inner edge of the shroud is provided with three notches for allowing the three tongues to be inserted therein. By this structure, the present invention can be assembled firmly, and the head strap can be tightened or loosened easily.

### BACKGROUND OF THE INVENTION

1. Field of the Invention

The present invention relates to a swimming goggle, in particular to a swimming goggle and a fastening assembly thereof.

2. Description of Prior Art

Swimming is a gentle and systemic exercise for completely stretching muscles of limbs and increasing cardiopulmonary function. Thus, swimming can avoid injuries of joints, muscles or bones caused by other fierce exercises (e.g. basketball). Usually, people like to wear a swimming goggle when swimming in water in order to have a clear vision in the water. Therefore, various swimming goggles have been proposed.

In general, the swimming goggle is constituted of an eyepiece frame, a head strap, and a fastening assembly. Since the user's head is different in size, the fastening assembly is configured to adjust the degree of tightness of the head strap around the user's head. In other word, the user utilizes the fastening assembly to tighten or loosen the head strap. In a common swimming goggle, the head strap is curvedly disposed through several slots provided in the fastening assembly. The desired length of the head strap can be obtained by the frictional force exerted by the tightened head strap to the slots of the fastening assembly. However, in such a structure, the head strap is merely fastened by the frictional force. When the user takes off the swimming goggle, the frictional force between the head strap and the fastening assembly is reduced, so that the previously-fastened position of the head strap relative to the fastening assembly may be changed due to any external force or an unintentional swing of the swimming goggle exerted by the user. As a result, the user may have to adjust the head strap again when wearing the swimming goggle in the next time.

A common swimming goggle available in the market has the following structure. Both ends of the eyepiece frame are provided with a through-hole for allowing one end of the head strap to be disposed through. Then, both ends of the head strap respectively pass through several guiding slots of the fastening assembly. After the head strap curvedly passes through the guiding slots, a stopper pin provided on the fastening assembly is inserted into one of adjusting holes formed on the head strap, thereby fixing the position of the head strap relative to the fastening assembly. Although the insertion of the stopper pin into the adjusting hole increases the fixing effect of the head strap, it is inconvenient for the user to take off the swimming goggle every time when he/she intends to adjust the degree of tightness of the head strap around the head.

Another conventional swimming goggle includes an eyepiece frame, eye glasses mounted in the eyepiece frame, and fasteners connected to both sides of the eyepiece frame. Each of the fasteners comprises a first cover, a second cover and an operating element connected between the first cover and the second cover. The first cover is provided with a guiding slot for allowing the head strap to be disposed through. The operating element has an abutting plate and an activating plate both of which are formed into an L shape and pivotally connected to the first cover and the second cover respectively. The operating element further comprises a driving arm axially extending from the activating plate as well as a first flexible arm and a second flexible arm obliquely extending from one end of the driving arm. The first flexible arm and the second flexible arm are provided with a pressing piece respectively. When the pressing pieces are pressed at the same time, the first flexible arm and the second flexible arm are subjected to the pressing force to generate an elastic deformation, which causes the driving arm to abut against the activating plate. As a result, the abutting plate moves away from the activating plate, thereby releasing the engagement between the abutting plate and the head strap. However, such a structure is so complicated that the flexible arms may suffer an elastic fatigue or overstretching due to a long-term use or an excessive force.

Therefore, it is an important issue for the present Inventor to solve the above-mentioned problems.

### SUMMARY OF THE INVENTION

The present invention is to provide a swimming goggle, which can be assembled easily as a firm construction and the degree of tightness of a head strap around the head can be adjusted easily.

The present invention provides a swimming goggle, including: an eyepiece frame having a connecting portion on both sides thereof, each of the connecting portions being provided with a through-holes; a head strap having a plurality of protrusions on both sides of its one surface; and two fastening assemblies for assembling each connecting portion with one end of the head strap, each of the fastening assemblies comprising: a fastening base connected with an operating arm, a surface of the operating arm having an engaging piece engaged between two adjacent protrusions, the periphery of the fastening base being formed with three tongues, the fastening base being provided with a locking hole corresponding to the through-hole and a trough located away from the locking hole for allowing the head strap to be disposed through; and a shroud assembled with the fastening base in such a manner that the connecting portion is sandwich between the shroud and the fastening base, one end of the shroud having a hook disposed through the through-hole and engaged with the locking hole, a surface of the shroud being provided with an adjusting opening for allowing the operating arm and the head strap to pass through, the inner edge of the shroud being provided with three notches for allowing the three tongues to be inserted therein.

The present invention is to provide a fastening assembly of a swimming goggle, which can be assembled easily as a firm construction and the degree of tightness of a head strap of the swimming goggle around the head can be adjusted easily.

The present invention provides a fastening assembly of a swimming goggle for assembling an eyepiece frame and a head strap of the swimming goggle, both sides of the eyepiece frame being provided with a through-hole, both ends of the head strap being formed with a plurality of protrusions, the fastening assembly comprising: a fastening base connected with an operating arm, a surface of the operating arm having an engaging piece engaged between two adjacent protrusions, the periphery of the fastening base being formed with three tongues, the fastening base being provided with a locking hole corresponding to the through-hole and a trough located away from the locking hole for allowing the head strap to pass through; and a shroud assembled with the fastening base, one end of the shroud having a hook disposed through the through-hole and engaged with the locking hole, a surface of the shroud being provided with an adjusting opening for allowing the operating arm and the head strap to pass through, the inner edge of the shroud being provided with three notches for allowing the three tongues to be inserted therein.

In comparison with prior art, the present invention has the following advantages:

According to the present invention, the periphery of the fastening base is formed with three tongues, and the inner edge of the shroud is correspondingly formed with three notches for allowing the three tongues to be inserted therein. Thus, the user can easily assemble the three tongues of the fastening base with the notches of the shroud. Thus, the present invention can be assembled easily.

According to the present invention, since the hook of the shroud passes through the through-hole of the eyepiece frame to be engaged in the locking hole of the fastening base, the shroud is assembled with the fastening base in such a manner that the connecting portion of the eyepiece frame is sandwiched between the shroud and the fastening base. Further, the three tongues of the fastening base are inserted into the three notches of the shroud, so that the present invention can be assembled together to form a firm construction.

According to the present invention, the fastening base is connected with the operating arm, and the surface of the operating arm is formed with an engaging piece. Further, the profile of the engaging piece is designed to have a vertical surface and a guiding slope opposite to the vertical surface. When the head strap passes through the trough of the fastening base and the adjusting opening of the shroud, the engaging piece of the operating arm is engaged between two adjacent protrusions of the head strap. The guiding slope of the engaging piece is oriented toward the trough, while the vertical surface is oriented toward the adjusting opening. With this arrangement, the user merely pulls the free-end section of the head strap protruding from the adjusting opening to tighten the head strap around the head without taking off the swimming goggle. When the user intends to loosen the heat strap, the user only needs to lift the operating arm and pulls the other section of the head strap by his/her finger. At this time, the protrusions of the head strap outside the fastening assembly can pass through the engaging piece to enter the adjusting opening smoothly. In this way, the user can adjust the degree of tightness of the head strap to fit his/her head without taking off the swimming goggle.

According to the present invention, since the engaging piece is engaged between two adjacent protrusions of the head strap, the vertical surface of the engaging piece can tightly abut against the protrusion to achieving a fastening effect. Such a fastening effect is not achieved by an elastic deformation of a flexible arm, so that the problem in prior art that the flexible arm may suffer an elastic fatigue to deteriorate the fastening effect of the fastening assembly can be avoided.

### BRIEF DESCRIPTION OF DRAWING

FIG. 1 is a partially exploded perspective view showing the swimming goggle of the present invention;

FIG. 2 is an exploded perspective view showing the fastening assembly of the present invention;

FIG. 3 is a partially assembled perspective view showing the swimming goggle of the present invention;

FIG. 4 is a partially assembled cross-sectional view showing the swimming goggle of the present invention;

FIG. 5 is a partially assembled cross-sectional view of the swimming goggle of the present invention, showing that the operating arm is lifted for adjusting the degree of tightness of the head strap; and

FIG. 6 is a partially assembled cross-sectional view of the swimming goggle of the present invention, showing that the operating arm is pressed for fixing the degree of tightness of the head strap.

### DETAILED DESCRIPTION OF THE INVENTION

The detailed description and technical contents of the present invention will become apparent with the following detailed description accompanied with related drawings. It is noteworthy to point out that the drawings is provided for the illustration purpose only, but not intended for limiting the scope of the present invention.

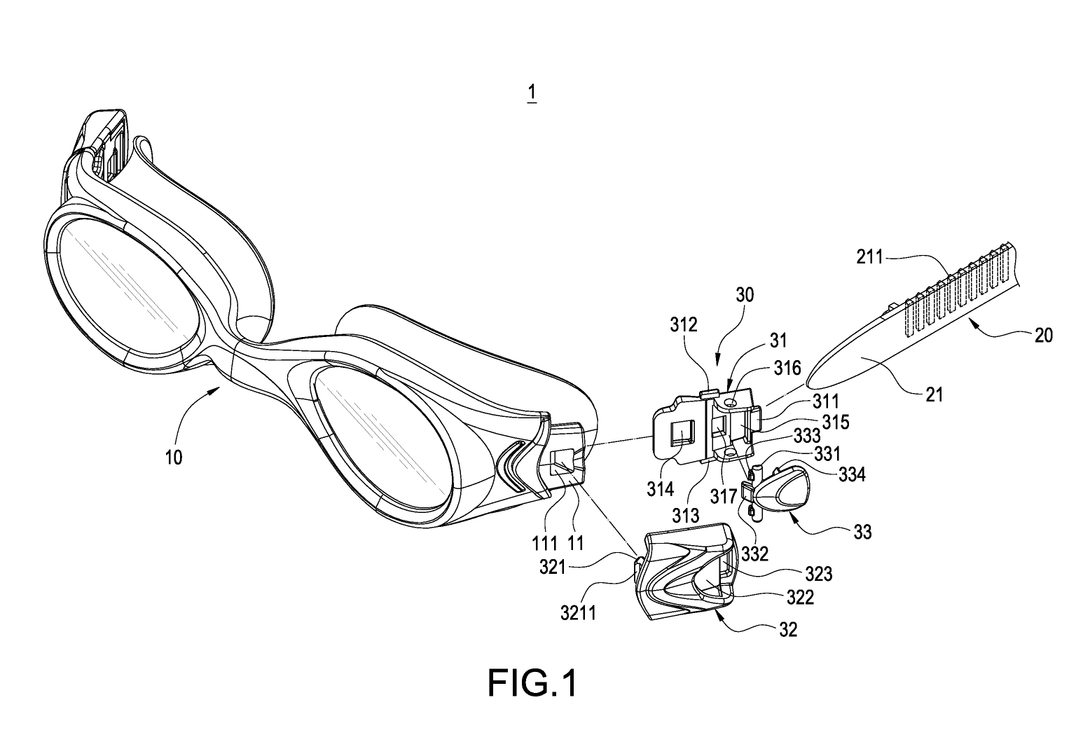

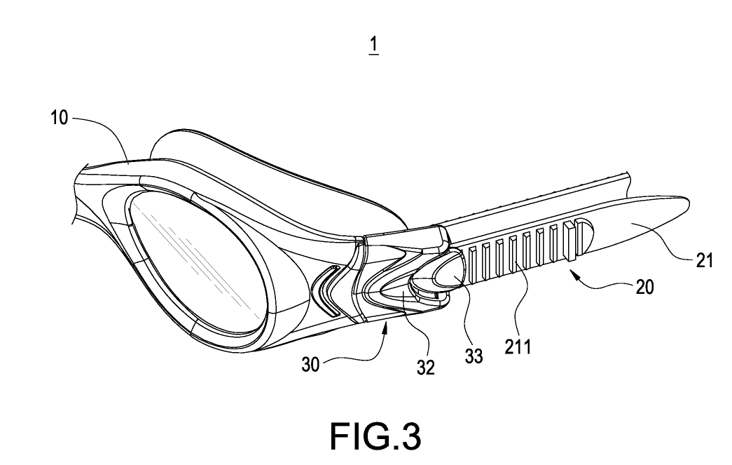

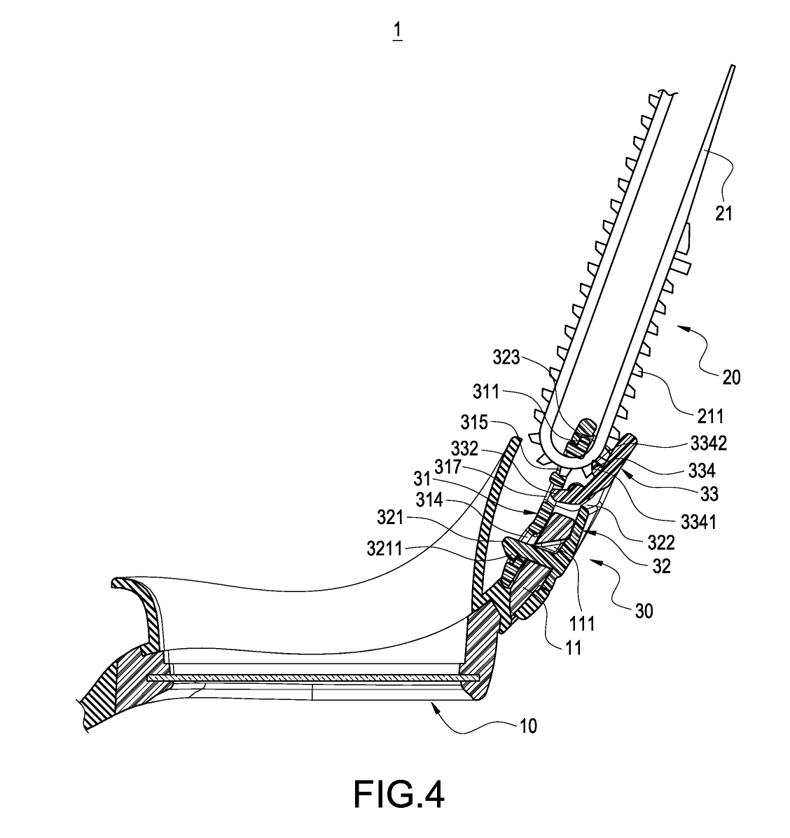

Please refer to FIGS. 1 to 4. The present invention provides a swimming goggle 1 including an eyepiece frame 10, a head strap 20 and two fastening assemblies 30.

Both sides of the eyepiece frame 10 are formed with a connecting portion 11 respectively for assembling with the fastening assemblies 30. The connecting portion 11 is provided with a through-hole 111. More specifically, the through-hole 111 is perpendicular to the extending direction of the connecting portion 11. As for the remaining portions of the eyepiece frame 10, since they are not the technical characteristics of the present invention, the description thereof is omitted for simplicity. Any suitable one-piece or two-piece eyepiece frame 10 may be used as long as both ends thereof are formed with a through-hole 111 respectively.

The head strap 20 is made of flexible plastic materials. Both ends 21 of the head strap 20 are formed with a plurality of protrusions 211. These protrusions 211 are arranged at intervals on one surface of the head strap 20 and vertically extend from the head strap 20. As for the remaining portions of the head strap 20, since they are not the technical characteristics of the present invention, the description thereof is omitted for simplicity. Any suitable types of head straps 20 may be used as long as both ends 21 thereof are formed with protrusions 211.

Each of the fastening assemblies 30 is used to assemble the connecting portion 11 with one end 21 of the head strap 20. The fastening assembly 30 comprises a fastening base 31 and a shroud 32.

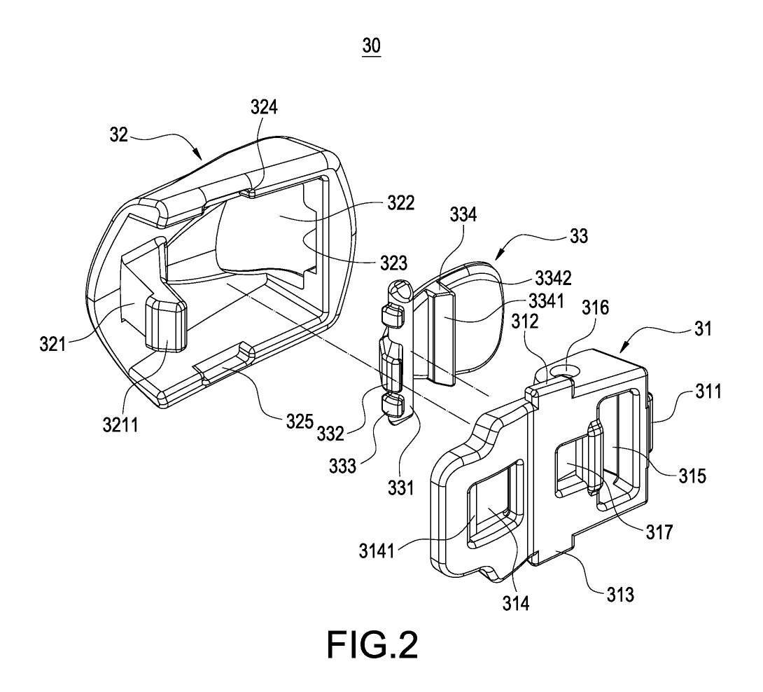

The fastening base 31 is connected with an operating arm 33. In the embodiment shown in FIG. 1, the operating arm 33 is pivotally connected to the fastening base 31. One end of the operating arm 33 is formed with a pivotal shaft 331. One side of the pivotal shaft 331 away from the operating arm 33 is formed with a restricting piece 332. The pivotal shaft 331 is formed with two restricting bosses 333 on both sides of the restricting piece 332. One surface of the operating arm 33 is formed with an engaging piece 334 engaged between two adjacent protrusions 211. As shown in FIG. 2, the engaging piece 334 has a guiding slope 3341 and a vertical surface 3342. The guiding slope 3341 is oriented toward the pivotal shaft 331, while the vertical surface 3342 is oriented away from the pivotal shaft 331.

The periphery of the fastening base 31 is formed with three tongues 311, 312 and 313. The fastening base 31 is provided with a locking hole 314 corresponding to the through-hole 111 and a trough 315 located away from the locking hole 314 and allowing the head strap 20 to pass through. Both sides of the fastening base 31 are provided with a shaft hole 316 respectively for allowing the pivotal shaft 331 of the operating arm 33 to be pivotally connected thereto. A restricting hole 317 is formed between the locking hole 314 and the trough 315 for allowing the restricting piece 332 of the operating arm 33 to swing therein. The two restricting bosses 333 are configured to restrict the moving distance of the operating arm 33 on the fastening base 31 along the pivotal shaft 331. The restricting piece 332 is configured to restrict the lifting angle of the operating arm 33. With this arrangement, the engaging piece 334 of the operating arm 33 can restrict the movement of the head strap 20 in the fastening assembly 3.

The shroud 32 is assembled with the fastening base 31 in such a manner that the connecting portion 11 is sandwiched between the shroud 32 and the fastening base 31. One end of the shroud 32 is formed with a hook 321 disposed through the through-hole 111 and engaged within the locking hole 314 of the fastening base 31. The shroud 32 is provided with an adjusting opening 322 for allowing the operating arm 33 and the head strap 20 to pass through. As shown in FIG. 2, the inner edge of the shroud 32 is provided with three notches 323, 324, 325 for allowing the three tongues 311, 312, 313 to be inserted therein.

It should be noted that the free end of the hook 321 is formed with a hooked portion 3211, and the bottom surface of the fastening base 31 is provided with a locking notch 3142 (FIG. 2) at the periphery of the locking hole 314. With this arrangement, when the hook 321 of the shroud 32 passes through the locking hole 314 of the fastening base 31, the hooked portion 3211 can be engaged within the locking notch 3141 on the bottom surface of the fastening base 31.

Moreover, the end of the fastening base 31 having the locking hole 314 has been engaged with the hook 321 of the shroud 32. Thus, the other end of the fastening base 31 has to be engaged with the shroud 32 to complete the assembly of the fastening base 31 and the shroud 32. To this end, the tongue 311 provided on the fastening base 31 adjacent to the trough 315 is located at a higher level than the other two tongues 312, 313. Further, the notch 323 of the shroud 32 corresponding to the tongue 311 is formed at a higher level than the bottom edge of the shroud 32, so that the tongue 311 can be supported on the notch 323 of the shroud 32. Therefore, the fastening base 31 is engaged with the shroud 32 by using the above-mentioned two supporting points. The other two notches 324 and 325 located on both sides of the adjusting opening 322 are formed on the bottom edge of the shroud 32. The two tongues 312 and 313 located on both sides of the operating arm 33 can be engaged within the notches 324 and 325 from the underside of the shroud 32.

With the above arrangement, the user can align the three tongues 311, 312, 313 of the fastening base 31 with the notches 323, 324, 325 of the shroud 32. More specifically, the user makes the tongue 311 adjacent to the trough 315 of the fastening base 31 to be supported in the notch 323 adjacent to the adjusting opening 322, and then assembles the fastening base 31 with the shroud 32 until the hook 321 of the shroud 32 passes through the locking hole 314 of the fastening base 31 and the hooked portion 3211 is engaged within the locking notch 3141 of the locking hole 314. When the user intends to detach the fastening base 31 from the shroud 32, he/she needs to pull the hooked portions 3211 out of the locking notch 3141 of the fastening base 31 to separate the shroud 32 from the fastening base 31. Thus, the fastening assembly 1 of the present invention can be assembled easily. Since four connecting points (located at the three tongues 311, 312, 313 and the hook 321) are formed between the fastening base 31 and the shroud 32, the fastening assembly 1 has a firm structure and cannot be disassembled easily.

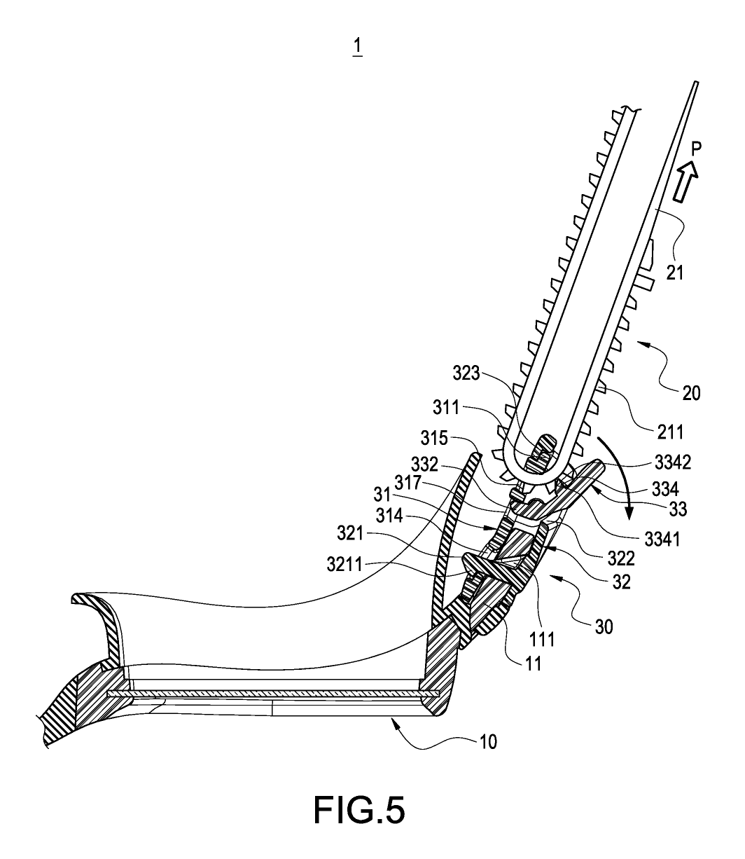

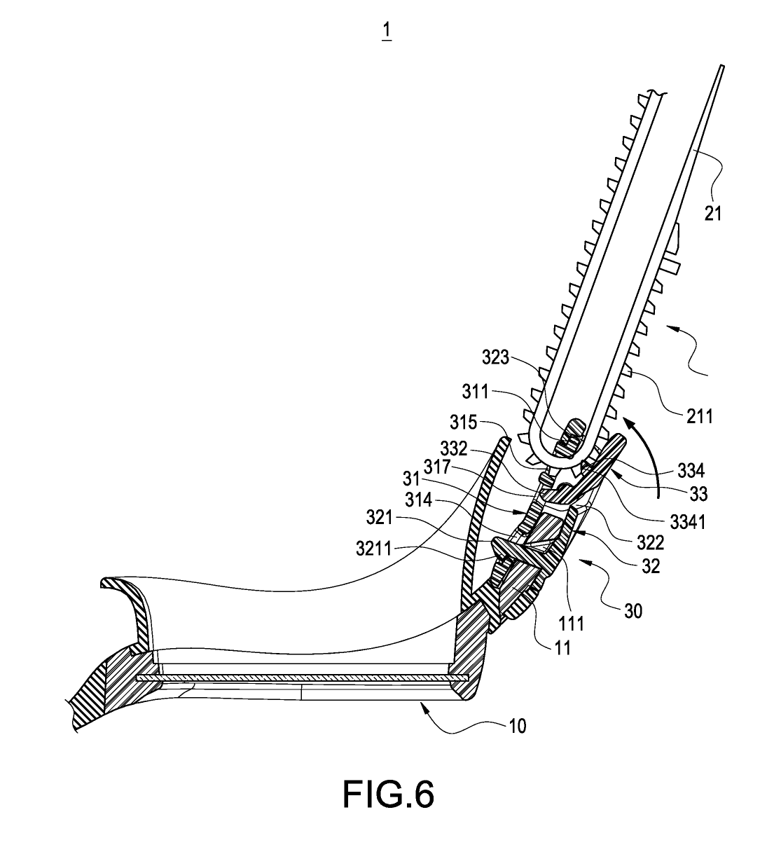

Next, the process of adjusting the degree of tightness of the head strap 20 around the head will be described with reference to FIGS. 5 and 6.

As shown in FIG. 5, one surface of the head strap 20 is formed with a plurality of protrusions 211. When the fastening assembly 30 is assembled with the connecting portion 11 of the eyepiece frame 10, one free end 21 of the head strap 20 is disposed through the trough 315 of the fastening base 31 and the adjusting opening 322 of the shroud 32 in such a manner that the engaging piece 334 of the operating arm 33 is engaged between two adjacent protrusions 211. Since the guiding slope 3241 of the engaging piece 334 is oriented toward the trough 315, the protrusions 211 of the head strap 20 can pass through the guiding slope 3341 of the engaging piece 334 easily. In other words, when the user pulls the free end 21 of the head strap 20, the protrusions 211 of the head strap 20 can pass through the guiding slope 3341 of the engaging piece 334 so as to slightly lift the operating arm 33 as indicated by the clockwise arrow in FIG. 5. Therefore, according to the present invention, when the user intends to tighten the head strap 20, he/she only needs to pull the free end 21 of the head strap 20 without taking off the swimming goggle 1.

As shown in FIG. 6, after the user adjusts the degree of tightness of the head strap 20 around the head, the user presses the operating arm 33 in a direction as indicated by the counterclockwise arrow in FIG. 6. At this time, the vertical surface 3342 of the engaging piece 334 abuts against one protrusion 211 to prevent the protrusion 211 from passing through the engaging piece 334, thereby fixing the position of the head strap 20 relative to the fastening assembly 30.

When the user intends to loosen the head strap 20, he/she lifts the operating arm 33 in a clockwise direction shown in FIG. 5 to make the operating arm 33 to be separated from the fastening base 31. At this time, the user pulls the section of the head strap 20 not passing though the trough 315 to move in a direction reverse to the arrow P, so that the protrusions 211 can pass through the engaging piece 334 to enter the fastening assembly 30. In other words, when the user intends to loosen the head strap 20, he/she lifts the operating arm 33 and pulls the section of the head strap 20 rather than its free end 21 (i.e. the section of the head strap 20 abutting against the head) without taking off the swimming goggle 1.

Although the present invention has been described with reference to the foregoing preferred embodiment, it will be understood that the invention is not limited to the details thereof. Various equivalent variations and modifications can still occur to those skilled in this art in view of the teachings of the present invention. Thus, all such variations and equivalent modifications are also embraced within the scope of the invention as defined in the appended claims. For example, the operating arm 33 may obliquely extend from one surface of the fastening base 31 rather than be pivotally connected thereto because the operating arm 33 integrally formed on the fastening base 31 still achieves the same effect as the above as long as the engaging piece 334 of the operating arm 33 is engaged between two adjacent protrusions 211. Accordingly, the pivotal shaft 331 and the restricting piece 332 of the operating arm 33, and the shaft holes 316 and the restricting hole 317 of the fastening base 31 may be omitted.

What is claimed is:

1. A swimming goggle, including:
an eyepiece frame having a connecting portion on both sides thereof, each of the connecting portions being provided with a through-holes;
a head strap having a plurality of protrusions on both sides of a surface thereof; and
two fastening assemblies for assembling each connecting portion with one end of the head strap, each of the fastening assemblies comprising:
a fastening base connected with an operating arm, a surface of the operating arm having an engaging piece engaged between two adjacent protrusions, the periphery of the fastening base being formed with three tongues, a surface of the fastening base being provided with a locking hole corresponding to the through-hole and a trough located away from the locking hole for allowing the head strap to pass through; and
a shroud assembled with the fastening base in such a manner that the connecting portion is sandwiched between the shroud and the fastening base, one end of the shroud having a hook disposed through the through-hole and engaged within the locking hole, a surface of the shroud being provided with an adjusting opening for allowing the operating arm and the head strap to pass through, an inner edge of the shroud being provided with three notches for allowing the three tongues to be inserted therein.

2. The swimming goggle according to claim 1, wherein a free end of the hook is formed with a hooked portion, a locking notch is formed at the periphery of the locking hole on the bottom surface of the fastening base, and the hooked portion is engaged with the locking notch.

3. The swimming goggle according to claim 2, wherein the tongue of the fastening base adjacent to the trough is located in a higher level than the other two tongues, the notch of the shroud adjacent to the adjusting opening is located in a higher level than the bottom edge of the shroud, the other two notches formed on both sides of the shroud are formed on the bottom edge of the shroud.

4. The swimming goggle according to claim 3, wherein the engaging piece has a guiding slope and a vertical surface, the guiding slope is oriented toward the trough, and the vertical surface is oriented away from the trough.

5. The swimming goggle according to claim 1, wherein the operating arm is pivotally connected to the fastening base, one end of the operating arm is formed with a pivotal shaft, both sides of the fastening base are formed with a shaft hole respectively for allowing the operating arm to be pivotally connected thereto.

6. The swimming goggle according to claim 5, wherein one side of the pivotal shaft away from the operating arm is formed with a restricting piece, the pivotal shaft is formed with two restricting bosses on both sides of the restricting piece, a restricting hole is provided between the locking hole and the trough for allowing the restricting piece to swing therein.

7. The swimming goggle according to claim 6, wherein a free end of the hook is formed with a hooked portion, a locking notch is formed at the periphery of the locking hole on the bottom surface of the fastening base, the hooked portion is engaged with the locking notch.

8. The swimming goggle according to claim 7, wherein the tongue of the fastening base adjacent to the trough is located in a higher level than the other two tongues, the notch of the shroud adjacent to the adjusting opening is located in a higher level than the bottom edge of the shroud, the other two notches formed on both sides of the shroud are formed on the bottom edge of the shroud.

9. The swimming goggle according to claim 8, wherein the engaging piece has a guiding slope and a vertical surface, the guiding slope is oriented toward the pivotal shaft, and the vertical surface is oriented away from the pivotal shaft.

10. The swimming goggle according to claim 1, wherein one free end of the head strap passes through the trough and the adjusting opening in such a manner that the engaging piece of the operating arm is engaged between two adjacent protrusions.

11. The swimming goggle according to claim 1, wherein the head strap is made of flexible plastic materials, and the protrusions are vertically arranged at intervals on one surface of the head strap.

12. A fastening assembly of a swimming goggle, configured for assembling an eyepiece frame with a head strap of the swimming goggle, both sides of the eyepiece frame being provided with a through-hole respectively, both ends of the head strap having a plurality of protrusions, the fastening assembly comprising:
a fastening base connected with an operating arm, a surface of the operating arm having an engaging piece engaged between two adjacent protrusions, the periphery of the fastening base being formed with three tongues, a surface of the fastening base being provided with a locking hole corresponding to the through-hole and a trough located away from the locking hole for allowing the head strap to pass through; and
a shroud assembled with the fastening base, one end of the shroud having a hook disposed through the through-hole and engaged with the locking hole, a surface of the shroud being provided with an adjusting opening for allowing the operating arm and the head strap to pass through, an inner edge of the shroud being provided with three notches for allowing the three tongues to be inserted therein.

13. The fastening assembly of a swimming goggle according to claim 12, wherein a free end of the hook is formed with a hooked portion, a locking notch is formed at the periphery of the locking hole on the bottom surface of the fastening base, and the hooked portion is engaged with the locking notch.

14. The fastening assembly of a swimming goggle according to claim 13, wherein the tongue of the fastening base adjacent to the trough is located in a higher level than the other two tongues, the notch of the shroud adjacent to the adjusting opening is located in a higher level than the bottom edge of the shroud, the other two notches formed on both sides of the shroud are formed on the bottom edge of the swimming goggle.

15. The fastening assembly of a swimming goggle according to claim 14, wherein the engaging piece has a guiding slope and a vertical surface, the guiding slope is oriented toward the trough, and the vertical surface is oriented away from the trough.

16. The fastening assembly of a swimming goggle according to claim 12, wherein the operating arm is pivotally connected to the fastening base, one end of the operating arm is formed with a pivotal shaft, both sides of the fastening base are formed with a shaft hole respectively for allowing the operating arm to be pivotally connected thereto.

17. The fastening assembly of a swimming goggle according to claim 16, wherein one side of the pivotal shaft away from the operating arm is formed with a restricting piece, the pivotal shaft is formed with two restricting bosses on both sides of the restricting piece, a restricting hole is provided between the locking hole and the trough for allowing the restricting piece to swing therein.

18. The fastening assembly of a swimming goggle according to claim 17, wherein a free end of the hook is formed with a hooked portion, a locking notch is formed at the periphery of the locking hole on the bottom surface of the fastening base, and the hooked portion is engaged with the locking notch.

19. The fastening assembly of a swimming goggle according to claim 18, wherein the tongue of the fastening base adjacent to the trough is located in a higher level than the other two tongues, the notch of the shroud adjacent to the adjusting opening is located in a higher level than the bottom edge of the shroud, the other two notches formed on both sides of the shroud are formed on the bottom edge of the swimming goggle.

20. The fastening assembly of a swimming goggle according to claim 19, wherein the engaging piece has a guiding slope and a vertical surface, the guiding slope is oriented toward the pivotal shaft, and the vertical surface is oriented away from the pivotal shaft.
